(19) United States
(12) Patent Application Publication
(10) Pub. No.: US 2025/0281697 A1
(43) Pub. Date: Sep. 11, 2025

(54) SENSOR ASSEMBLY FOR DETECTING OR RECOGNIZING OPERATION AN INJECTION DEVICE

(71) Applicant: Sanofi, Paris (FR)

(72) Inventor: Michael Helmer, Frankfurt am Main (DE)

(21) Appl. No.: 18/862,475

(22) PCT Filed: May 2, 2023
(86) PCT No.: PCT/EP2023/061437

§ 371 Date: Nov. 1, 2024

(30) Foreign Application Priority Data
May 3, 2022 (EP) 22315093.9

(51) Int. Cl. A61M 5/315

(52) U.S. Cl. CPC A61M 5/31568; A61M 5/3157

(57) ABSTRACT

The disclosure relates to a sensor assembly for detecting an operation of an injection device, the sensor assembly including a sensor element attachable to the injection device and including a sensing surface, the sensing surface having a number of touch sensitive sensor segments the touch sensitive sensor segments being operable to generate an electric touch signal when touched by a body part of a user, and a processor connectable to the sensor element, operable to detect a variation of the electric touch signals over time and to recognize an operation of the injection device on the basis of the temporal variation of the electric touch signals.

### CROSS-REFERENCE TO RELATED APPLICATIONS

The present application is the national stage entry of International Patent Application No. PCT/EP2023/061437, filed on May 2, 2023, and claims priority to European Application No. EP 22315093.9, filed on May 3, 2022, the disclosures of which are incorporated herein by reference.

### TECHNICAL FIELD

The present disclosure relates to a sensor assembly for detecting or recognizing and operation of an injection device. In another aspect the disclosure relates to an injection device equipped with such a sensor assembly. In a further aspect the disclosure relates to an add-on device configured for fastening to an injection device, wherein the add-on device is provided with a sensor assembly. In another aspect the disclosure relates to a method of detecting or recognizing an operation of an injection device an in a further aspect the disclosure relates to a computer program.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is desirable to provide an automated detecting and logging of a repeated and regular use of the drug delivery device. Especially for fixed dose injection devices typically offering a simple and rather effective approach for delivery of many drug therapies, recording of doses injected by a user would offer a significant advantage over a manual dose logging in terms of security and convenience.

Some injection devices or add-on devices configured for use with injection devices offer an electronic detection and monitoring of repeated dose injection procedures. Here, a user may require some training for correctly using such a detection or monitoring function. It is hence a particular aim to provide an intuitive and rather easy monitoring or logging of an operation of an injection device.

### SUMMARY

The present disclosure relates to a sensor assembly for detecting, recognizing, characterizing and/or measuring an operation of an injection device. The sensor assembly includes a sensor element attachable to the injection device and having a one-dimensional or two-dimensional sensing surface. The sensing surface includes a number of touch sensitive sensor segments. The sensor assembly further includes a processor connectable to the sensor element. The processor is operable to detect a sensing area on the sensing surface when in contact with a body part of a user.

The processor is operable to detect a variation of the sensing area over time and the processor is further operable to recognize, to detect, to characterize and/or to measure an operation of the injection device on the basis of the detected variation of the sensing area.

The sensing area is typically defined by a portion of the sensing surface of the sensor element that is in contact with the body part of the user. Insofar, the sensing area may define or may coincide with a contact area or touch area of the sensing surface that is in contact with the body part, e.g. with a finger of a user.

By monitoring a temporal variation of the sensing area during e.g. a single and/or a predefined operation of the injection device the processor is operable to characterize, to recognize, to detect and/or to measure a particular operation of the injection device.

In some embodiments, each of the sensor segments is operable to generate or to modify an electric touch signal when touched by the body part of the user. The sensor segments are spatially separated on the sensing surface in a non-overlapping way. They may be arranged next to each other in a regular or irregular manner. In some examples, the entire sensing surface is covered and/or occupied by numerous sensor segments. The sensor segments may be of equal or of unequal size.

In some examples, the touch sensitive sensor segments are decoupled from each other. Each touch sensitive sensor segment may operate independent from any other touch sensitive sensor segment of the sensing surface. Hence, then touch sensitive sensor segments may be operable to generate electric touch signals individually when touched by the body part of the user.

The sensor segments may belong to a touch sensitive matrix or may constitute a touch sensitive matrix, e.g. a one or two-dimensional array of touch sensitive segments. The touch sensitive segments may each include a capacitor or resistor, that is operable to generate or to modify an electrical signal in response to a physical contact, e.g. with a body part of a user. Insofar, the plurality of touch sensitive sensor segments forms or constitutes a spatially resolving touch sensitive sensor.

In accordance with further examples the touch sensitive surface or touch sensitive matrix includes a matrix of electrically resistive elements, which change their electrical and measurable resistance when e.g. touched by a user. In other examples, the touch sensitive surface or touch sensitive matrix includes a matrix of capacitive elements operable to change their measurable electric capacitance when e.g. touched by a user.

In further examples the touch sensitive surface or touch sensitive matrix comprises a combination of electrically resistive sensor segments and capacitive sensor segments. Capacitive sensors may exhibit a rather low degree of electric power consumption.

In further examples, the touch sensitive surface or touch sensitive matrix is based on surface acoustic wave technology that relies upon soundwaves. Accordingly, the touch sensitive surface or touch sensitive matrix includes at least one pair of an acoustic wave transducer and an acoustic wave receiver.

In another example the touch sensitive surface or touch sensitive matrix comprises a number of optical sensors, such as photodetectors or photodiodes.

In further examples, the touch sensitive surface or touch sensitive matrix includes ultrasonic sensors. Optical sensors and/or ultrasonic sensors could be also implemented as fingerprint sensors being capable to distinguish a characteristic fingerprint of a first user from a characteristic fingerprint of a second user.

In some examples, the sensor element is attachable to a user actuatable portion of the injection device. It may be attachable or may be attached to a surface of an actuation element or handling element, which actuation element or handling element is usually touched, handled or actuated by a user when using or operating the injection device. The sensor element may be also integrated into a surface of the actuation element or handling element. The user actuatable portion may be a portion of at least one of a sleeve-shaped housing component, a trigger or a dose dial of the injection device.

In some examples, the processor is operable to detect a point of time, at which an injection is started. The processor may detect a point of time at which an injection procedure terminates. The processor may be further operable to detect or to measure a duration during which the sensor element or the sensing surface is in contact with a body part of the user. Insofar, the processor may be operable to measure or to determine a duration of operation of the injection device. Moreover, by evaluating and processing the temporal variations of the electric touch signals and/or or by monitoring and/or evaluating the temporal variation of the sensing area the processor and hence the sensor assembly may be operable to distinguish between different operation modes of the injection device.

Depending on the temporal variation of the electric touch signals and/or depending on the variation of the sensing area the processor may be operable to automatically distinguish e.g. between a dose setting procedure and a dose dispensing or dose injection procedure.

In some examples, the processor may be further operable to detect or to characterize a holding operation of the injection device. Here, the processor may be operable to detect and/or to monitor a time interval or a particular duration at the end of a dose injection procedure during which the injection needle should remain in the skin of the patient.

According to a further example at least one of the touch sensitive sensor segments is operable to generate different electric touch signals in response to a variation of a pressure applied to the touch sensitive sensor segment. Insofar, the electric touch signals to be generated by a touch sensitive sensor segment vary with the pressure e.g. applied by a body part of a user getting in touch or getting in contact with this particular touch sensitive sensor segment.

In response to a variation of the pressure applied to the touch sensitive sensor segment the electric touch signal may differ in magnitude or amplitude. Furthermore, the electric touch signal may change its sign or may change its frequency or periodicity. In response to a varying pressure applied to the touch sensitive sensor segment the respective sensor segment is operable to vary the electric touch signal in a measurable way, i.e. in a way that is detectable or processable by the processor connected or connectable to the touch sensitive sensor segment.

In some examples, numerous touch sensitive sensor segments are operable to generate different electric touch signals in response to a variation of the pressure applied thereto. With some examples, the touch sensitive sensor segments of the sensing surface are operable to provide and/or to generate a variation of respective electric touch signals in response to a variation of the pressure applied to the respective touch sensitive sensor segments.

In some examples, the touch sensitive sensor segment is operable to generate at least two different electric touch signals in response to a variation of a pressure applied thereto. Per default the touch sensitive sensor segment may be operable to generate a first electric touch signal in response to a first pressure applied to the touch sensitive sensor segment. Here, a first electric touch signal may be generated if the first pressure applied to the touch sensitive sensor segment is above a first predefined threshold.

The touch sensitive sensor segment may be further operable to generate a second electric touch signal that differs in at least one of a magnitude, an amplitude, a sign or a frequency from the first electric touch signal. The second electric touch signal may be generated when the pressure applied to the touch sensitive sensor segment is at or above a second predefined threshold. Typically, the second threshold is above the first threshold.

Insofar, the first and second electric touch signals may be indicative of a rather low and a rather high pressure applied to the respective touch sensitive sensor segments, respectively.

According to a further example, the touch sensitive sensor segment or a plurality of sensor segments are operable to generate a variety of different electric touch signals. It is conceivable that the touch sensitive sensor segment is operable to generate at least three, at least four, at least five, at least six or at least eight different electric touch signals, each of which reflecting or indicating a respective first, second, third, fourth, fifth, sixth or eights pressure applied to the respective touch sensitive sensor segment. The touch sensitive sensor segment may be operable to generate an electric touch signal that varies gradually with a variation of the pressure applied to the respective touch sensitive sensor segment. Here, the electric touch signal may be directly representative of the applied pressure. Also, the touch sensitive sensor segment of the sensing surface is operable to generate different electric touch signals in response to a variation of the pressure applied to the respective touch sensitive sensor segments. In some examples, all touch sensitive sensor segments of the sensing surface are operable to generate different or varying electric touch signals in response to a variation of the pressure applied to the respective touch sensitive sensor segments.

The touch sensitive sensor segments can be arranged adjacently or next to each other on the one-dimensional or two-dimensional sensing surface of the sensor element the entire sensing surface may become touch sensitive.

Depending on the number and size of touch sensitive sensor segments distributed across the sensing surface there can be provided a precise and rather detailed spatially resolved pressure profile of or across the sensing surface of the sensor element. In this way, the sensor assembly is operable to detect or to measure a spatially resolved pressure profile applied across the sensing surface by a body part of a user.

The spatially resolved pressure profile and/or a temporal variation of such a spatially resolved pressure profile may be indicative of a particular operation of the injection device.

According to a still further example the processor is operable to process electric touch signals of a number of touch sensitive sensor segments to ascertain a sensing area of the sensing surface being touched by the body part. Typically, and when using a finger as a body part to get in contact with the sensing surface of the sensor element the respective touch sensitive sensor segments will be able to detect a pressure applied by the respective body part. In this way all touch sensitive sensor segments getting in mechanical contact with the body part during or for operation of the injection device may generate a respective electric touch signal.

By simultaneously processing the signals of the touch sensitive sensor segments the processor may provide or may ascertain a sensing area on the sensing surface that is actually in touch or in mechanical contact with the body part of the user. In this way, the processor is operable to detect, whether a middle part or a border region of the sensing surface is actually touched by the body part.

Moreover, the processor may be operable to detect or to measure a size of the sensing area. By ascertaining or determining a sensing area of the sensing surface being actually touched or being in mechanical contact with the body part of the user the sensor assembly provides a rather precise and spatially resolved monitoring of how the body part of the user touches the sensor element.

According to a yet further example, the processor is operable to detect a movement of the sensing area on the sensing surface. In situations, in which the body part of the user itself is subject to a movement during the operation of the injection device, e.g. during setting of a dose or during injection of a dose the movement of the body part e.g. relative to a housing of the injection device may be accompanied by a measurable movement of the body part relative to the sensor element and hence relative to the sensing surface. Here, the body part of the user, e.g. a finger or a thumb may be subject to a milling motion or a natural rolling motion during the operation of the injection device.

Such a movement of the body part relative to the sensor element may lead to a movement of the sensing area on the sensing surface that is touched by the body part. A movement of the body part relative to the sensor element may induce an activation and deactivation of numerous touch sensitive sensor segments spatially distributed across the sensing surface of the sensor element. This leads to a measurable change of a variation of electric touch signals and to a respective variation of the sensing area measurable by the processor.

By evaluating and processing the electric touch signals generated by the touch sensitive sensor segments during a respective operation of the injection device the processor may detect the movement of the sensing area on the sensing surface. By detecting such a movement of the sensing area the sensor assembly may recognize or characterize an operation of the injection device. Here, the sensor assembly and hence its processor may be operable to distinguish between different operation modes of the injection device by evaluating a measurable movement of the sensing area on the sensing surface.

According to a yet further example the processor of the sensor assembly is operable to detect a variation of a size of the sensing area on the sensing surface. A variation of a size of the sensing area may result from a variation of the pressure applied by the body part onto the sensor element. Since the body part may include a certain elasticity and may further include a rather convex-shaped outside facing structure or surface, by increasing a pressure applied by the body part onto the sensor element the fraction of the body part getting in direct contact with the sensing surface may increase, e.g. due to an elastic deformation of the body part. This may lead to an increase of the size of the sensing area on the sensing surface.

A detection of a variation of the size of the sensing area during an operation of the injection device may be hence indicative for a variation of the pressure applied by the user onto the sensor element. In some examples, the size variation measurable by the processor as well as a variation of the type, magnitude, amplitude, sign or frequency of the electric touch signals generated by the touch sensitive sensor segments may be processed in a combined manner. Insofar, a variation of the pressure applied by the body part to the sensor element may be detected by a variation of the size of the sensing area concurrently with a variation of the electric touch signals generated by the touch sensitive sensor segments.

Here, a variation of the pressure applied by the body part may be monitored or detected in a twofold manner. Hence, a variation of a pressure applied to the sensor element can be measured or determined in at least two different ways thereby increasing or providing a redundancy of the measurement system provided by the sensor assembly.

According to a further example, the processor is operable to detect a variation of a geometry and/or to detect a variation of an orientation of the sensing area on the sensing surface. In this way, further modes of operation, during which the body part of the user is subject to a movement or change relative to the sensor element can be detected and evaluated. Variations of a geometry or orientation of the sensing area on the sensing surface, which is typically detected by a respective variation of electric touch signals generated by a number of touch sensitive sensor segments may be further indicative of particular scenarios of use of the injection device.

By electronically detecting and/or by electronically and quantitatively measuring at least one of a movement of the sensing area, a variation of a size of the sensing area, a variation of a geometry and/or a variation of an orientation of the sensing area on the sensing surface the sensor assembly is operable to detect, to recognize, to characterize and/or to measure different operations and operation modes of the injection device. By evaluating a movement of the sensing area during operation of the injection device and/or by evaluating a variation of the size, geometry or orientation of the sensing area on the sensing surface while the injection device is operated by a user the sensor assembly may automatically recognize a particular operation mode of the injection device.

According to a further example, the sensor element includes a planar sensing surface configured for fastening to an end face of a trigger of the injection device. The planar sensing surface may include a number of touch sensitive sensor segments located adjacently next to each other on the planar sensing surface. With some examples the entire planar sensing surface may be filled or occupied with adjacently arranged sensor segments. Insofar, the entire planar sensing surface may be implemented as a touch sensitive sensing surface of the sensor element.

The planar sensing surface may cover the entirety of an end face of a trigger of the injection device. Typically, the trigger is configured to be depressed by a finger of a user to initiate and/or to control an injection procedure conducted by the injection device. By applying or attaching the planar sensing surface on the depressible end face of the trigger the sensor element and hence the entire sensor assembly may be integrated into the trigger of the injection device.

In another example, the planar sensing surface may be configured for fastening to an end face of an auxiliary trigger of an add-on device configured for fastening to an injection device. The auxiliary trigger may mimic the trigger of the injection device and may operably engage with the trigger of the injection device when the add-on device is attached or fastened to the injection device. Here, the auxiliary trigger may replace or substitute the functionality of the original trigger of the injection device.

The add-on device may cover the trigger section of the injection device. The auxiliary trigger may be in direct or indirect operable engagement with the trigger of the injection device, such that by depressing or actuating the auxiliary trigger of the add-on device the trigger of the injection device will be actuated, respectively. With an implementation of the sensor assembly to or into an add-on device the planar sensing surface may be fastened or integrated to an end face of such an auxiliary trigger of the add-on device.

In some examples, the planar sensing surface may be integrated into the end face of the trigger or auxiliary trigger. It may be fastened to the end face of the trigger or may be integrated into the end face of the trigger. By attaching or integrating the planar sensing surface to the end face of the trigger there can be provided a rather integrated solution for implementing or integrating the sensor assembly into or onto the injection device or at an add-on device.

According to a further example, the sensor element includes a tubular shaped sensing surface configured for fastening to a tubular member of the injection device. With this example the sensor element may be configured to enclose or to at least partially enclose a tubular shaped structure or tubular member of the injection device, such as a dose dial or a housing component of the injection device.

In some examples, the tubular shaped sensing surface of the sensor element is configured to enclose an outside surface of the tubular shaped dose dial of the injection device. A dose of individual size of the injection device may be set by the user using the dose dial and rotating the dose dial either in a dose incrementing direction or dose decrementing direction relative to the body or housing of the injection device. By having the tubular shaped sensing surface mounted or integrated on the outside of the dose dial such a dose dialing procedure can be precisely detected, recognized, characterized or even quantitatively measured by the sensor assembly.

In other examples, the tubular shaped sensing surface is attachable or fixable to an outside surface of a tubular shaped housing component of the injection device. The housing component may be clasped by a hand, e.g. by a palm or by numerous fingers of a user of the injection device for preparing and/or for conducting a dose injection procedure. Also here, and by having the sensor element on the outside surface of the tubular shaped body or housing of the injection device the user induced handling or operation of the injection device can be precisely detected, characterized, recognized or measured.

According to a further example, the sensor element includes a planar sensing surface and further includes a tubular shaped sensing surface. The sensor element may include a cup-shaped structure comprising the planar sensing surface at a longitudinal end adjoining a tubular-shaped sensing surface forming a sidewall of the cup-shaped sensor element. Such a sensor element may be operable or configured for attaching to a dose member of an injection device, wherein the dose member is implemented as a combined dose dial and trigger. Here, by rotating the dose member a dose of variable size may be set and by depressing the dose member the injection process may be triggered and/or controlled.

According to a further example, the sensor element includes a flexible sheet configured for wrapping around the tubular member of the injection device. The sensor element may include a planar substrate made of a flexible sheet, e.g. made of a flexible foil. The planar substrate may be pliable and/or foldable or wrappable into a tubular shape.

The flexible sheet or substrate may be provided with numerous touch sensitive sensor segments. The pliable or flexible sheet enables to wrap around the tubular member of the injection device. A flexible sheet of the sensor element and the touch sensitive sensor segments attached or mounted on the flexible sheet allows to use the sensor element with a large variety of differently sized tubular members of the injection device.

The sensor element may be universally applicable to tubular members of different size. By way of the flexible sheet the entire sensor element may be flexible and may be easily fixable to the tubular member of the injection device. It may be adhesively attached to an outside surface of the tubular member and may provide a spatially resolving touch sensitive area on the outside of the tubular member, e.g. on the outside of the housing of the injection device.

The same may also apply to an add-on device presumed that the add-on device includes a tubular member, e.g. for fastening or clasping around the housing of the injection device.

According to a further example, the tubular member of the injection device is a dose dial rotatable relative to a body of the injection device for setting of a dose. By attaching the flexible sensor element to the dose dial a dose dialing or dose setting operation conducted by a user of the injection device, thereby rotating the dose dial relative to the body or housing of the injection device can be detected, recognized, characterized or quantitatively measured by the sensor assembly.

According to a further example, the tubular member is a body of the injection device. With some examples the body of the injection device includes an elongated tubular sleeve. The body may be sized to accommodate a drive mechanism for expelling or withdrawing a dose of a medicament from a medicament container. With some examples the medicament or medicament container, e.g. implemented as a syringe or cartridge containing the liquid medicament, may be also arranged inside the body or housing of the injection device.

According to a further example, the tubular member is a clip configured for detachably fastening to the injection device. Here, the tubular member may be implemented as a part of an add-on device configured for fastening to the housing of the injection device.

In a further example of the sensor assembly, the processor is operable to detect at least one of a dose setting operation of the injection device, a dose dispensing operation of the injection device and a holding operation of the injection device by processing numerous electric touch signals of numerous touch sensitive sensor segments over time when the sensor element is attached to the injection device or when the sensor element is indirectly attached to the injection device, e.g. by an auxiliary device or add-on device configured for fastening to the injection device.

In some examples, the processor is operable to detect at least one of a dose setting operation, a dose dispensing operation or holding operation of the injection device by processing temporal variations of the sensing area of the sensing surface of the sensor element.

In a further example, and when a dose setting operation is accompanied by a rotation of the tubular member of the injection device, e.g. of a dose dial, such a rotation can be detected by a variation of electric touch signals provided by touch sensitive sensor segments that are provided on the tubular shaped sensing surface of the sensor element. A dispensing operation, e.g. the start, the duration and/or the end of a dispensing operation can be typically detected, recognized or characterized by processing of electric touch signal generated or modified by touch sensitive sensor segments of the planar sensing surface, which is typically provided on an end face of the trigger of the injection device or on the respective end face of an auxiliary trigger of an add-on device.

Moreover, an end of dose holding operation of the injection device, i.e. a period of time, during which the user should apply and maintain a certain pressure on the trigger of the injection device after termination of the dose injection procedure, can be precisely monitored. Here, and during the holding operation at the end of a dose injection procedure the electric touch signals generated or modified by the numerous touch sensitive sensor segments should be rather constant.

According to some examples, the processor is operable to distinguish between a dose setting operation, a dose dispensing operation and an end of dose holding operation of the injection device by processing numerous electric touch signals of numerous touch sensitive sensor segments over time when the sensor element is attached to the injection device or to the add-on device, which add-on device itself is then attached to the injection device. Each operation mode of the injection device can be characterized by a temporal and/or spatial profile of numerous touch sensitive sensor segments being touched by the body part of the user during the respective operation mode.

By evaluating such a temporal and/or spatial profile measurable by the sensor assembly the sensor assembly is and becomes operable to automatically distinguish between different operation modes of the injection device. Insofar, the sensor assembly can be provided with an automated operation mode detection of the injection device thus simplifying the use of the sensor assembly and increasing the precision of a continuous injection monitoring or injection logging over time.

According to a further example, the sensor assembly includes a clock or a clock generator connected to the processor. This way, the processor is operable to detect or to measure at least one of a point of time, at which a user operates the injection device, and a duration during which a user operates the injection device. By providing the processor with a clock, respective points of time or time durations can be detected or logged, at which the processor detects a particular electric touch signal or a variation of a sensing area being indicative e.g. of a start or termination of at least one of a dose setting operation and a dose dispensing operation.

According to a further example, the sensor assembly includes a memory connected or coupled to the processor. The processor is operable to store use related data in the memory, wherein the data contains at least one of a point of time at which the injection device was operated, the duration during which the device was operated and a size of a dose of the medicament set or injected by the injection device. All these parameters, points of time, time durations and dose size information can be derived from a variation of the electric touch signals or on the basis of a variation of the sensing area detectable by the processor connected to the sensor element.

According to a further example, the processor, the clock and the memory may be operable to automatically store or to automatically monitor and/or to log a sequence of user-induced operations of the injection device only by detecting or measuring variations of electric touch signals or variations of the sensing area of the sensor element.

According to a further example, the processor may be provided with a wake-up function. Here, the processor may automatically switch into a sleep mode, e.g. when determining that the sensor element and its sensing surface has not been touched over a predefined time interval. Upon detection of an initial contact with the sensing surface of the sensor element the processor may switch into an activated mode and may wake up accordingly. By providing the processor with a sleep functionality energy provided to the sensor assembly can be saved. Battery lifetime of the sensor assembly can be hence prolonged accordingly.

According to a further example, the sensor assembly includes a power source, e.g. in form of an electric power source, such as a battery.

In further examples the sensor assembly is provided with a transceiver. The transceiver may be operable to communicate with an external electronic device, e.g. with a smartwatch, a smart phone or a tablet computer. The transceiver may be implemented as a wireless transceiver. The transceiver may be operable to establish a communication link with the external electronic device. With some examples the transceiver may be operable to harvest energy from the external electronic device. Insofar, the transceiver may also serve as a kind of a power source for providing electric power to the sensor assembly.

According to a further example, the sensor assembly includes a signal generator. The signal generator may be implemented as one of a visual signal generator, an acoustic signal generator or a haptic signal generator. By way of the signal generator the sensor assembly may be operable to directly communicate with a user of the injection device. This way, the sensor assembly may provide a confirmation or the like feedback to the user, thus indicating, that e.g. a particular user-induced operation of the injection device has been monitored or has not been monitored correctly. The signal generator may be operable to provide and/or to generate visual signals of different color and/or of different and varying durations.

In other examples, the signal generator may be operable to generate an acoustic signal, such as an audible sound. Here, the signal generator may be operable to provide different and distinguishable sound signals, e.g. indicating a successful or unsuccessful detection or measuring of a user-induced action of the injection device.

The haptically implemented signal generator may be configured to produce a vibration that is detectable, e.g. palpable by a user.

In further examples, the sensor assembly may include a reminder functionality. Here, the memory of the sensor assembly may be provided with a predefined medication schedule for a particular patient. Accordingly, and when an injection may be due, the processor may prompt the signal generator to generate a user perceivable signal thus reminding the user to conduct or to execute an injection procedure.

In further examples, the sensor assembly includes a position sensor and/or an acceleration sensor. The position sensor may be integrated into the sensor assembly or may be integrated into the injection device. The position sensor may be operably connected with a component of the drive mechanism of the injection device being indicative of a size of a dose currently set or dispensed. The position and/or orientation of the position sensor may be further indicative of an amount of medicament provided in the cartridge. In this way the position sensor may provide quantitative data being indicative of a size of a dose.

In further examples and when provided with an acceleration sensor, the sensor assembly may further detect or measure a particular movement or acceleration of the sensor assembly, e.g. being indicative of a particular gesture conducted by a user when holding the sensor assembly. By way of the acceleration sensor an operation mode of the injection device may be detected accordingly.

By way of the transceiver, the sensor assembly may exchange measured data with the external electronic device. The memory of the sensor assembly is typically configured to store use-related data of the injection device. When establishing a communication link with the external electronic device the sensor assembly, hence the memory of the sensor assembly may synchronize with the external electronic device, which device may provide further processing of the data and/or a transmission of the data, e.g. to a healthcare provider.

In some examples, the sensor assembly may be provided with a display, e.g. operable to visually indicate use-specific or user-specific information to a user of the injection device. The display may be implemented into the sensor element. Insofar, the sensor element may be implemented as a touch sensitive display.

Here, the touch sensitive sensor segments may represent individual pixels of the touch sensitive display. The touch sensitive sensor segments may coincide with the pixels of the touch sensitive display.

The sensor element, and in particular its sensing surface may be provided with a reconfigurable electronic display, e.g. operable to provide visual content to a user of the add-on device or injection device, respectively. Insofar, the sensor element and the sensor assembly provides a twofold function. It is operable to receive input from a user as well as to provide information to the user.

In some examples, the sensor assembly could be operable to provide a visual indication on the sensing surface, such as a number of doses actually set or a number of doses to be set. The sensor assembly could support a patient during setting of a dose as well as during injection of a dose. The sensor assembly, and in particular the touch sensitive display thereof may be operable to provide visual symbols, numbers and/or text in order to guide and/or to assist a user in using of the add-on device or injection device.

The touch sensitive display of the sensor assembly may further provide instructions to the user, such as to set a dose, to inject a dose, to confirm an injection, to submit data between the add-on device and an external electronic device and/or to inform the user when the next injection will be due.

Moreover, the touch sensitive display may be operable to visually indicate a particular portion on the sensing surface that should be touched by the user.

The touch sensitive display may provide static information as well as dynamic information. In some examples, the touch sensitive display of the sensor assembly may be operable to dynamically and visually illustrate a motion of a finger of a user across the sensing surface of the sensor assembly. Here, the user may be even guided of how touch, tip or swipe across the sensing surface of the sensor element.

According to a further example of the sensor assembly includes a clock and a memory connected to the processor. The processor is operable to monitor a temporal variation of electric touch signals during a specific operation of the injection device one. The processor is further operable to store the temporal variation of the electric touch signals as a touch signal profile in the memory and to assign the touch signal profile with the specific operation of the injection device.

This operation of the sensor assembly or functionality of the sensor assembly may represent a training mode. Here, a user may train the sensor assembly to assign or to map a measurable or derivable touch signal profile to one of a number of user specific operations of the injection device.

Insofar, the sensor assembly may be switchable between a training mode and a sensing mode. This way, the sensor assembly may be individually adapted to individual and different modes of operation of different users of injection devices. The sensor assembly may be hence adaptable to different user-specific habits and behaviors, which may be different for each user of an injection device.

According to a further example, the processor is operable to detect a temporal variation of electric touch signals during an operation of the injection device and to generate an operation profile on the basis of the detected temporal variations of electric touch signals. The processor is further operable to compare the operation profile with numerous touch signal profiles previously stored in the memory. The processor is further operable to select one touch signal profile from the memory having a best match with the actually measured operation profile and to select the specific operation of the injection device previously assigned with the selected touch signal profile as a recognized operation of the injection device.

Here, the processor and hence the sensor assembly is operable to make use of touch signal profile previously stored in the memory. Individually trained touch signal profiles may be used to assign and to map an actually measured operation profile with a predefined operation or specific operation of the injection device. In this way, the sensor assembly and/or its processor is operable to distinguish between different operation modes of the injection device simply by comparing an actually measured operation profile with a previously stored touch signal profile.

In another aspect, the present disclosure also relates to an injection device for injecting a dose of a medicament. The injection device includes a body to accommodate a drive mechanism operable to withdraw or to expel the medicament from a medicament container. Typically, the drive mechanism is operable to inject or to expel a dose of the medicament from the medicament container. The injection device further includes at least one of a dose dial and a trigger which is actuatable by a user for injecting and/or for setting of a dose.

The injection device further includes a sensor assembly as described above. The sensor assembly is attached to or is integrated into at least one of the body, the dose dial and the trigger of the injection device. By way of attaching or integrating the sensor assembly to or into at least one of the body, the dose dial and the trigger there can be provided a rather precise and quasi-automated detection, recognition, characterization and measurement of user induced operations of the injection device.

The injection device may be implemented as a pen-type injector. It may be implemented as a disposable injection device or as a reusable injection device. With some examples the injection device may include a dial extension, which is subject to a longitudinal and rotative or helical motion during setting of a dose and which is subject at least to a longitudinal sliding movement during dose injection. With other examples the injection device is void of a so-called dial extension. Here, a dose dial and/or trigger may be provided e.g. at a proximal end of the housing of the injection device. The dose member, e.g. in form of a combined dose dial and trigger may be rotatable relative to the body or housing of the injection device for setting of a dose and may be depressible, e.g. by a thumb of a user for injecting the dose.

In other examples, the injection device is implemented as a so-called autoinjector. Here, the user may simply hold the body of the injection device and press the body and hence a distal end of the injection device against a portion of a skin, thereby inducing a rather automated injection procedure, during which an injection needle is urged into the skin and a dose of the medicament is subsequently dispensed or injected.

According to a further aspect, the present disclosure also relates to an add-on device configured for fastening to an injection device. The add-on device includes a device body and a fastener for fastening the device body to at least one of a body, a dose dial and a trigger of the injection device. The add-on device includes a sensor assembly as described above. Insofar, the entire functionality of the sensor assembly may be integrated into the add-on device. Typically, and when correctly attached to the injection device the add-on device provides setting and/or injecting of a dose, e.g. by providing or comprising at least one of an auxiliary dose dial and/or an auxiliary trigger operably engageable with the dose dial and/or with the trigger of the injection device when the add-on device is correctly assembled or attached to the injection device.

Insofar, all features, effects and benefits described above in connection with the sensor assembly equally apply to the injection device and to the add-on device, respectively.

According to another aspect, the present disclosure also relates to a method of recognizing, detecting, characterizing and/or measuring an operation of an injection device. The method includes the steps of attaching or integrating a sensor element or sensor assembly as described above to or into one of an injection device and an add-on device as described above, wherein the add-on device is configured for fastening to the injection device or to another injection device, e.g. being void of a sensor assembly as described above.

The method further includes the step of generating a number of electric touch signals in response to a body part of a user touching the sensing surface of the sensor element. Alternatively, the method includes the step of detecting or ascertaining a sensing area of the sensing surface being touched or being in contact with the body part of the user.

As a further step, the method includes a detecting of a variation of electric touch signals over time and recognizing, detecting, characterizing or measuring an operation of the injection device on the basis of the temporal variation of the electric touch signal(s).

Alternatively, and with another example the method includes the step of detecting a variation of the sensing area over time and detecting, recognizing, characterizing or measuring an operation of the injection device on the basis of the temporal variation of the sensing area.

Generally, the method of detecting, recognizing, characterizing and/or measuring operation of the injection device is to be conducted by an injection device as described above and/or or by using an add-on device as described above. Particularly, the method is to be conducted by a sensor assembly as described above. Insofar, all features, effects and benefits described above in connection with the sensor assembly, the injection device and/or the add-on device equally apply to the method of detecting, recognizing, characterizing answers or measuring the operation of the injection device; and vice versa.

In a further aspect, the present disclosure also relates to a computer program comprising computer readable instructions, which when executed by a processor of a sensor assembly as described above cause the processor to detect a variation of electric touch signals over time, which electric touch signals are generated by touch sensitive sensor segments of a sensing surface of a sensor element of the sensor assembly when touched by a body part of a user. The computer program further causes the processor to detect, to recognize, to characterize and/or to measure an operation of the injection device on the basis of the temporal variations of the electronic touch signal.

According to a further example, the computer program may be configured or may be operable to cause the processor to detect a variation of a sensing area of the sensing surface being touched by the body part and to detect, to recognize, to characterize and/or to measure an operation of the injection device on the basis of the temporal variation of the sensing area measurable by the sensor assembly.

The present disclosure further contemplates a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further contemplates a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure contemplates a data carrier having a data structure stored thereon, which, after loading into a processor, computer or computer network, such as into a working memory or main memory of the processor, computer or computer network, may execute the method according to one or more of the examples disclosed herein.

The present disclosure further contemplates a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

In another example, the present disclosure contemplates a modulated data signal which contains instructions readable by a processor, a computer system or computer network, for performing the at least parts of a method according to one or more of the examples disclosed herein. Preferably, referring to the computer-implemented aspects of the disclosure, one or more of the method steps or even all of the method steps of the method according to one or more of the examples disclosed herein may be performed by using a processor, a computer or computer network. Thus, generally, any of the method steps including gathering, provision and/or manipulation of data may be performed by using a processor, a computer or computer network. Generally, these method steps may include any of the method steps, typically except for method steps requiring manual work, such as providing the samples and/or certain aspects of performing the actual measurements.

Specifically, the present disclosure further discloses: A computer or computer network comprising at least one processor, wherein the processor is adapted to perform the method according to one of the examples described in this description, a computer loadable data structure that is adapted to perform the method according to one of the examples described in this description while the data structure is being executed on a processor, a computer, a computer program, wherein the computer program is adapted to perform the method according to one of the embodiments described in this description while the program is being executed on a computer.

Generally, the scope of the present disclosure is defined by the content of the claims. The disclosure is not limited to specific embodiments or examples but includes any combination of elements of different embodiments or examples. Insofar, the present disclosure covers any combination of claims and any technically feasible combination of the features disclosed in connection with different examples or embodiments.

In the present context, the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 schematically illustrates an on device configured for fastening to the injection device,

FIG. 3 schematically illustrates a cross-section through an example of an add-on device or an example of a cross-section through a portion of an injection device,

FIG. 4 shows a block diagram of an electronic module for implementing a sensor assembly,

FIG. 5 schematically shows an example of a sensor assembly in a perspective illustration,

FIG. 6 shows the sensor assembly of FIG. 5 in a side view,

FIG. 7 shows the sensor assembly of FIG. 5 from above,

FIG. 8 shows an example of using the sensor assembly,

FIG. 9 dose and example of a sensing area of the sensor assembly,

FIG. 10 shows another example of a sensing area,

FIG. 11 shows another example of a sensing area,

FIG. 12 shows a further example of a sensing area,

FIG. 13 shows a further example of a sensing area,

FIG. 14 shows a further example of a sensing area,

FIG. 15 shows a further example of a sensing area,

FIG. 16 shows a further example of a sensing area during use of the sensor assembly,

FIG. 17 schematically illustrates another mode of operation of the injection device,

FIG. 18 shows a further example of using the injection device,

FIG. 19 shows an operation mode of the injection device for thing of a dose,

FIG. 20 schematically illustrated a sensing area of the sensor assembly during the operation of the injection device as illustrated in FIG. 19,

FIG. 21 shows a further example of a sensing area during operation of the injection device according to FIG. 19,

FIG. 22 schematically illustrates a body part of a user,

FIG. 23 schematically shows another example of a sensor assembly configured for fastening to an outside surface of a tubular member of an injection device,

FIG. 24 schematically illustrates an operation of the injection device before injecting of a dose,

FIG. 25 schematically illustrates numerous sensing area of the sensor assembly in accordance to the operation mode of FIG. 24,

FIG. 26 schematically illustrated use of the injection device during and/or for injecting of a dose,

FIG. 27 schematically illustrated numerous sensing area of the sensor assembly during the operation mode of FIG. 26,

FIG. 28 illustrates an example of an external electronic device configured for communicating with the sensor assembly and

FIG. 29 is illustrative of a flowchart of a method of detecting, recognizing, characterizing or measuring an operation of an injection device.

### DETAILED DESCRIPTION

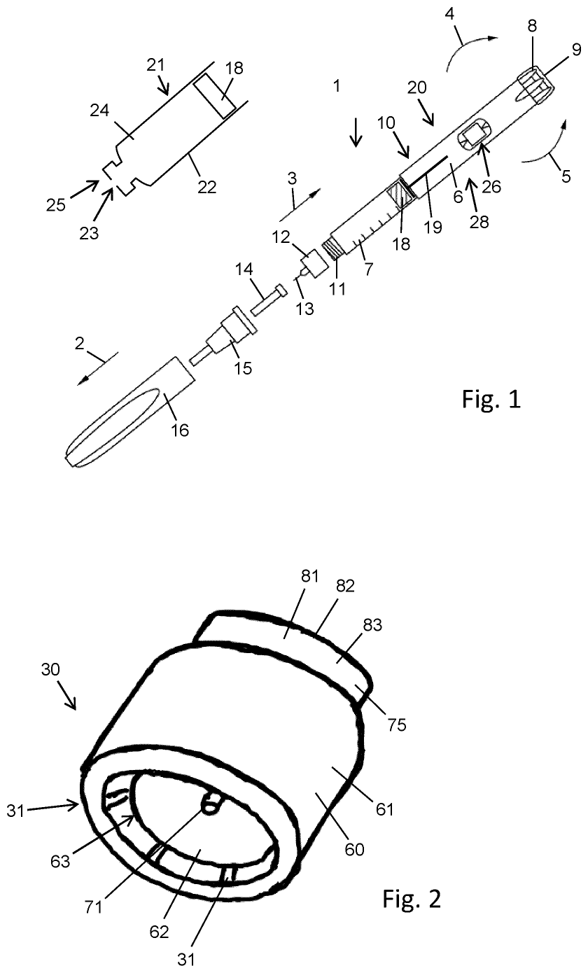

FIG. 1 shows an example of a drug delivery device 1, which is implemented as a handheld injection device. The injection device 1 may include or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 includes a dispensing end for dispensing or injecting the medicament 24. Towards the proximal direction 3 the injection device 1 includes at least one of a dose member 8 and a trigger 9, by way of which a dose of equal or individual or different size can be set and dispensed, respectively.

The injection device 1 includes a housing 10. The housing 10 may include numerous housing components, such as a body 6 and a cartridge holder 7. The body 6 may be sized and configured to accommodate a drive mechanism 20. The cartridge holder 7 is sized and configured to accommodate a medicament container 21, e.g. implemented as a cartridge containing the liquid medicament 24. The medicament container 21 includes a tubular-shaped barrel 22 sealed towards the distal end by a seal 23. The seal 23 may include a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the cartridge holder 7. The drive mechanism 20 of the injection device 1 includes a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism includes a mechanical energy storage configured to provide at least a portion of the dispensing force. Examples of drive mechanism can be found e.g. in WO 2004/078241 A1, WO 2014/033197 A1 or WO 2014/033195 A1 the entirety of which are herein incorporated by reference.

With some examples, as e.g. described or shown in FIGS. 8 and 18 the injection device 1 and hence the drive mechanism 20 may include a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the cartridge holder 7 has to be connected with a needle assembly 12. For this, the distal end of the cartridge holder 7 includes a connector 11, e.g. in form of a threaded interface to engage with a complementary shaped threaded counter interface of the needle assembly 12.

The needle assembly 12 is detachably or releasably fixable to the cartridge holder 7. It includes a double-tipped injection needle 13. A proximal end of the injection needle (not shown) is configured to enter into a through opening at the distal end face of the connector 11 or cartridge holder 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The cartridge holder 7 and hence a portion of the housing 10 is to be received in a protective cap 16, which is detachably connectable to the cartridge holder 7 or body 6.

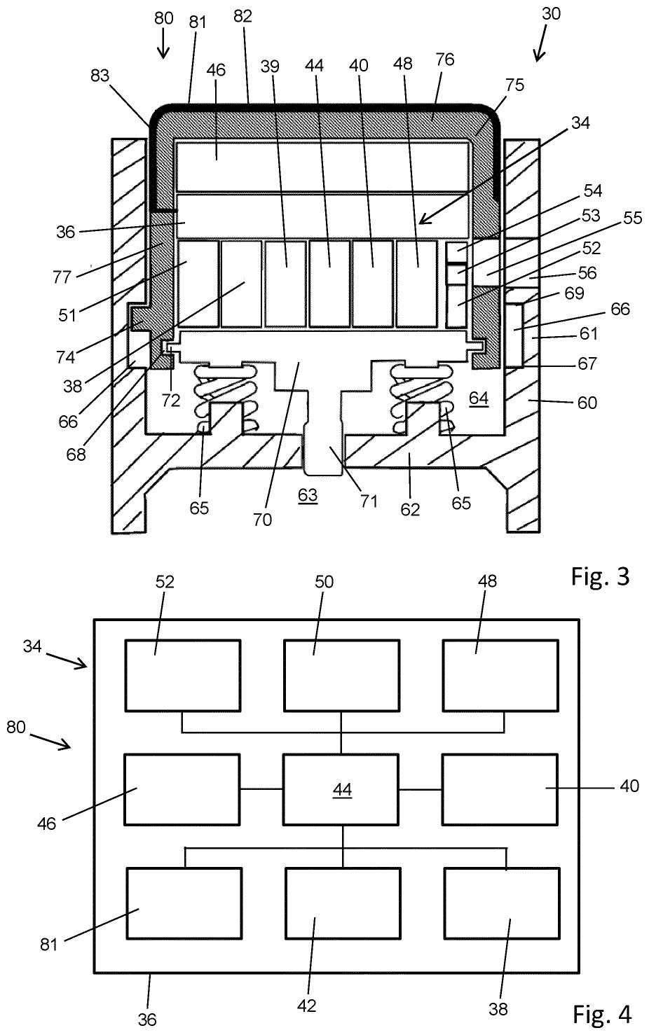

In FIGS. 2 and 3 there is shown an example of an add-on device 30 configured for fastening to the proximal end of the injection device 1. The add-on device 30 includes a sensor assembly 80 with a sensor element 81, which is operable to detect, to recognize, to characterize and/or to measure an operation of the injection device.

The add-on device 30 as illustrated in FIGS. 2 and 3 is detachably connectable to the dose dial 8. It includes a device body 60 with a tubular-shaped sidewall 61. Towards the distal end the sidewall 61 confines a receptacle 63, which is sized to receive the dose dial 8 and the trigger 9 of the injection device. For this, the inside of the sidewall 60 may include one or numerous fastening ribs 31, which are configured to provide a slip free fastening of the add-on device 32 the dose dial 8.

The receptacle 63 is confined by a radially inwardly extending flange portion 62. The flange portion 62 separates the interior of the device body 60 into a distal receptacle 63 and a proximal receptacle 64. The proximal receptacle 64 is sized to accommodate a support 70 and a cover 75 to house an electronic module 34. The support 70 includes a longitudinally extending stem 71 extending through the flange portion 62 in distal direction. The support 70 is displaceably mounted inside the receptacle 64. It is movable against the action of a return element or several return elements 65 in distal direction 2. The support 70 is connected with a cup-shaped cover 75, which protrudes in proximal direction from the sidewall 61 of the device body 60.

The cover 75 includes a planar-shaped end face 76 facing in proximal direction 3. The end face 76 is provided with the sensor element 81. As illustrated in FIG. 3 the entirety of the end face 76 may be covered by the sensor element 81. In this way, the end face 76 serves as an actuation surface to be depressed by a user, e.g. by a thumb 114 of a user. The user may apply a distally directed force onto the end face 76, thereby urging the cover 75 and the support 70 in distal direction 2 against the action of the return element 65.

Upon releasing the end face 76, the return element 75, e.g. implemented as a return spring, serves to move the support 70 and the cover 75 towards the proximal starting position as illustrated in FIG. 3. At least one of the cover 75 and the support 70 includes a radially outwardly extending projection 74 guided in a longitudinally extending recess 66 on the inside of the sidewall 61 of the device body 60. As seen in longitudinal direction the recess 66 is confined by a proximal stop face 69 and a distal stop face 67. In the initial configuration as illustrated in FIG. 3 the projection 74 is in longitudinal abutment with the proximal stop face 69 of the recess 66.

When depressing the cover 75 in distal direction 2 against the action of the return element 65, a distal end position is reached when the projection 74 engages the distal stop face 67. By way of the projection 74 guided in the groove 66 the longitudinal movement of the support 70 and the cover 75 can be delimited within predefined margins relative to the device body 60.

When depressed in distal direction 2, the support 70 and its stem 71 starts to protrude from the flange portion 62. Since the sidewall 61 is fixable in longitudinal direction to the dose dial 8 the distally directed longitudinal displacement of the support 70 and its stem 71 serves to act on the trigger 9, thereby inducing a distally directed motion of the trigger 9 relative to the dose dial 8 or body 6. The cover 75 is secured to the support 70 with regard to the longitudinal direction. It may be freely rotatable relative to the support 70, in particular during a dose injection procedure. During dose injection the dose dial 8 may be subject to a rotation in the dose decrementing direction 5 while the trigger 9 remains rotationally locked to the dial extension 27 and/or to the body 6. Here, the support 70 and/or the cover 75 may form or constitute an auxiliary trigger of the add-on device 30

Here, the inside of a sidewall of the cover 75 may include at least one of a radial protrusion and a recess 68 to engage with a complementary-shaped radial recess or protrusion 72 of the support 70. The cup-shaped cover 75 connected or fastened to the support 70 provides a receptacle for an electronic module 34 arranged inside the hollow space formed by the support 70 and the cover 75.

The sensor assembly 80 as described herein may be somewhat identical or equivalent to the electronic module 34; and vice versa. The electronic module 34 may include a printed circuit board 36. It may further include a transceiver 38, 39, a memory 40, a clock 42, a processor 44, a power source 46, an acceleration sensor 48, a position sensor 50, signal generator 51, 52 and e.g. light sources 53, 54. It may also include a microphone.

Moreover, a sidewall 77 of the cover 75 may include a window 55 aligned with a respective window 56 in the sidewall 61 of the device body 60. This way, visual signals producible by different light sources 53, 54 located inside the hollow space of the cover 75 can be perceived and visually detected from outside the add-on device 30.

The two light sources 53, 54 may belong to a visual signal generator 52, that is operable to produce or to generate visual signals of different color and/or of variable duration. The windows 55, 56 may be provided with a light pipe or with a light guiding structure. This way, the device body 60 can be protected against ingress of dust or humidity.

In FIG. 4, there is shown a block diagram of one example of the electronic module 34 and/or of the sensor assembly 80. As illustrated also in FIG. 3 the processor 44 is mounted on the printed circuit board 36, which is connected to the sensor element 81 of the sensor assembly 80 substantially covering the end face 76 of the cover 65.

In general, the electronic module 34 can be mounted on the printed circuit board 36. The electronic module 34 may be configured to communicate with an external electronic device 100, as e.g. illustrated in FIG. 28. The external electronic device 100 may be implemented as a mobile electronic device. It may include a smartwatch, a smart phone or a tablet computer. The electronic module 34 includes a transceiver 38, which is configured to establish or to built-up a communication link between the external electronic device 100 and the electronic module 34. A respective communication link can be realized in a wireless or wired manner.

The electronic module 34 may be configured to exchange data with the external electronic device 100. Data being indicative of an operation of the injection device and gathered or collected by the sensor assembly 80 may be transmitted to the external electronic device via the transceiver 38. The transceiver 38 may be implemented as a Bluetooth transceiver or as a BLE transceiver. The further transceiver 39 may be implemented as a NFC transceiver. The two transceivers may distinguish with regard to their communication protocol and/or with regard to their spatial range.

The electronic module 34 and hence the sensor assembly 80 includes a memory 40 configured to store a plurality of measurement results of the sensor assembly 80. The electronic module 34 and hence the sensor assembly 80 further includes a clock 42 configured to provide each of a plurality of measurement data of the sensor assembly 80 with a time index indicating the point of time and/or date of the detection of the respective operation of the injection device 1.

The processor 44 of the sensor assembly 80 and hence of the electronic module 34 is configured to control operation of the sensor assembly 80 and hence operation of the sensor element 81. The sensor assembly 80 and hence the electronic module 34 is further equipped with a power source 46 configured to power the processor 44 and the sensor assembly 80. The power source 46 may be implemented as a battery. The sensor assembly 80 may further include an acceleration sensor 48 configured to detect an injection operation of the injection device 1 and/or to detect or to classify gestures of a user when using the sensor assembly 80 and/or the injection device 1. For example, and when implemented as an autoinjector, the acceleration sensor may detect an acceleration of a needle of the autoinjector upon executing a dispensing or injection procedure.

The sensor assembly 80 may further include a position sensor 50, which may be operable to detect the position or orientation of a dedicated component of the drive mechanism 20 of the injection device 1. The position sensor may be operable to detect e.g. the position of a last dose nut, of a piston rod 19 or the like component of the drive mechanism 20 being indicative of the amount of medicament located inside the medicament container 21.

Moreover, the sensor assembly 80 or electronic module 34 includes a signal generator 52, which may be implemented as a visual signal indicator comprising at least one light source 53, 54. Additionally or alternatively, the signal generator 51 may be implemented as a haptic signal generator, e.g. configured to generate a perceivable vibration of the electronic module 34. With further examples the signal generator 51 may include an audible signal generator, configured to generate an audible sound.

In addition and in further examples, the electronic module 34 and/or the sensor assembly 80 may include a microphone, by way of which characteristic click noises of the injection device can be detected and processed, thereby allowing to derive or to measure a size of a dose currently set or injected by the injection device 1.

The presently illustrated implementation of the electronic module 34 in an add-on device 30 is only exemplary. Especially with reusable devices it is also conceivable that the entire functionality of the add-on device 30 and hence the entire functionality of the sensor assembly 80 and the electronic module 34 is implemented in an injection device 1. Here, the receptacle 64, which is sized to accommodate the hardware components of the electronic module 34 may be provided inside the dose dial 8, which is covered or closed in proximal direction by the trigger 9. Here, the trigger 9 of the injection device replaces the cover 75 and the support 70. The stem 71 of the support 70 and hence of the trigger 9 may be then operably engaged with the drive mechanism in order to induce or to trigger a dispensing operation thereof. The device body 60 may be provided or represented by the tubular shaped those the dial 8.

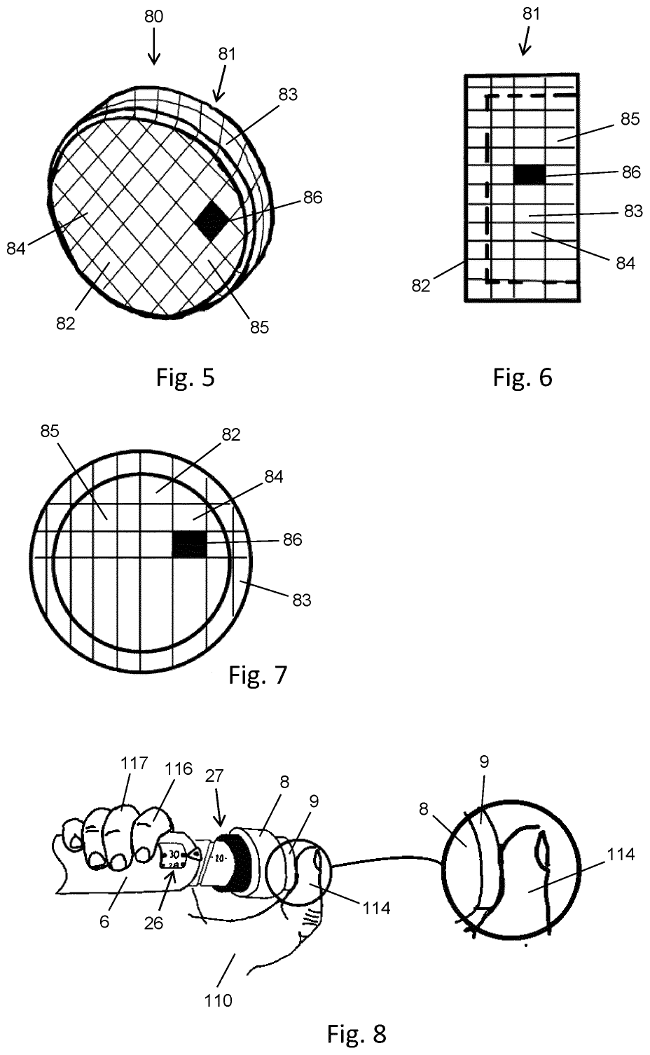

In FIGS. 2, 3 and 5-8 one example of a sensor assembly 80 is schematically illustrated. The sensor assembly 80 includes a sensor element 81 with numerous touch sensitive or pressure sensitive sensor segments 84, 85, 86 on a sensing surface 82, 83. The sensor assembly 80 may include a regular arrangement of touch sensitive sensor segments 84, 85, 86 as illustrated in FIGS. 5 and 6. The individual sensor segments may be comparable to pixels of a touch sensitive display. The sensor segments 84, 85, 86 may each include a capacity measuring device by way of which a mechanical contact with a body part of a user can be precisely detected.

In some examples, the sensor segments 84, 85, 86 are pressure sensitive. They are hence operable to generate or to modify an electric touch signal when touched by a body part of a user. The sensor segments 84, 85, 86 may be spatially distributed across the sensing surface 82, 83 of the sensor element 81. The sensor element 81 may provide a spatially resolved detection of a sensing area 88, 89 that is actually touched by or which is in mechanical contact with the skin of a user.

In some examples, the individual sensor segments 84, 85, 86 are not only touch sensitive but are also operable to generate electric touch signals being indicative of a pressure or strength of the mechanical contact with the body part of the user. Hence, the touch sensitive sensor segments 84, 85, 86 are operable to generate different electric touch signals being indicative of the size of a pressure or force applied to the respective sensor segment.

Insofar, the touch sensitive sensor segments 84, 85, 86 are operable to generate electric touch signals that differ in at least one of a magnitude, an amplitude, a sign or frequency. Those differences in the electric touch signals are detectable by the processor 44 of the sensor assembly 80 and can be evaluated so as to detect, to recognize, to characterize and/or to measure a user-induced operation of the injection device 1.

In the example as shown in FIGS. 5-8, the sensor element 81 includes a planar-shaped sensing surface 82 of circular shape and a tubular shaped sensing surface 83, which is located longitudinally adjacent to the outside circumference of the sensing surface 82. Insofar, the sensor element 81 includes a cup-shaped receptacle, which is sized e.g. to receive the trigger 9 of the injection device 1 and/or the cover 75 of the add-on device 30.

In further examples, it is conceivable that the cup-shaped sensor element 81 is configured to receive a dosing element, which combines the functionality of a dose dial 8 and a trigger 9 in a single part.

In the illustration of FIGS. 5-7, only one sensor segment 86 of the plurality of sensor segments 84, 85, 86 is active and detects a contact with a body part of a user.

In some examples, the sensor element 81 only includes a planar-shaped sensing surface 82 fastened to the proximal end face of a trigger 9 or of a cover 75 of an add-on device 30. With other examples the sensor element 81 may exclusively include a tubular shaped sensing surface 83 configured to confine a tubular-shaped member 28 of the injection device 1. The tubular shaped member 28 may be provided by the tubular shaped dose dial 8 or by the tubular shaped body 6 of the injection device 1.

In FIG. 8, a typical scenario of use of the injection device 1 provided with a dial extension 27 is illustrated. For setting of a dose the user rotates the dose dial 8 relative to the body 6. The dose dial 8 is a part of the dial extension 27, which then starts to project in proximal direction 3 from the proximal end of the body 6 as a dose of increasing size is set or dialed.

As a consequence, an increasing number of dose indicating indicia shows up in the dose indicating window 26. For injecting of a dose the user uses his hand 110 and clasps the body 6 of the injection device with his palm 112 thereby using at least two fingers 116, 117 to hold the body 6 of the injection device 1. The user may then use his thumb 114 to induce a dispensing action by depressing the trigger 9 in distal direction 2.

Since the trigger 9 and hence the entire dial extension 27 is subject to a distally directed longitudinal displacement relative to the body 6 during dose injection the angle at which the thumb 114 is oriented relative to the trigger 9 will be subject to a gradual change. Hence, during dispensing of the dose the thumb 114 is subject to a milling motion or natural rolling motion, which is detectable by the spatially resolved touch sensitive sensing surface 82 of the sensor element 81 covering the proximal face of the trigger 9 or of the cover 75 of the add-on device 30.

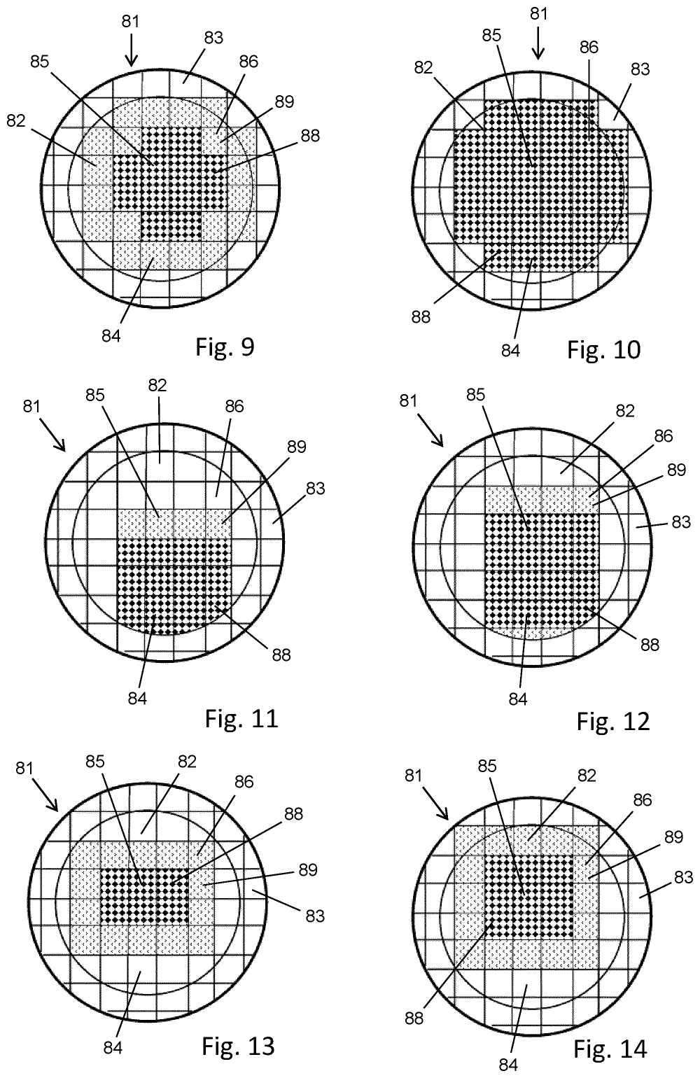

Moreover and as illustrated in greater detail in the FIGS. 9-16, the touch sensitive sensor segments 84, 85, 86 are operable to generate different electric touch signals dependent on the force level or pressure level applied to the respective sensor segments 84, 85, 86. In FIGS. 9-16 a high or a comparatively high pressure level is indicated by comparatively dark shaded sensor segments 84, 85, 86. A comparatively low pressure level is indicated by relatively weak or brightly shaded sensor segments 84, 85, 86.

In this way there is not only provided a spatially resolved contact profile across the one-or two-dimensional sensing surface 82, 83 of the sensor element 81 but the pressure profile may be also indicative of different pressure levels applied across the sensing surface(s) 82, 83.

In the example of FIG. 9, there are altogether only 12 sensor segments on the sensing surface 82 that are subject to a comparatively large force or pressure effect. Here, only the exemplary sensor segment 85 is subject to a comparatively high pressure. The sensor segments 84, 86 are located radially outside the sensing area 88 of high-pressure. They are subject to a moderate or comparatively low pressure.

The sensor segments 84, 86, which are subject to a comparatively low or moderate pressure form or constitute a sensing area 89 of low or moderate pressure. Those sensor segments 85 that are subject to a comparatively high pressure form or constitute a sensing area 88.

As illustrated in FIG. 9, the sensing area 88 is located almost in the middle of the sensing surface 82. It is surrounded by the sensing area 89 of comparatively low pressure. This example may represent a case, where a user applies a moderate pressure onto the sensing surface 82, e.g. with his thumb 114. Since the thumb 114 is of convex shape and is of a certain elasticity by increasing the pressure the thumb 114 of the user will be subject to a respective deformation, thus increasing the lateral extent of the sensing area 88 of high-pressure. As indicated in FIG. 10 those sensor segments 84, 86, which were previously only subject to a comparatively low pressure are now subject to a comparatively high pressure.

A change of the pressure profile from the example of FIG. 9 towards the example of FIG. 10 can be precisely detected and monitored by the processor 44, which is configured to individually process the electric touch signals produced or modified by each of the sensor segments 84, 85, 86. Insofar, the processor 44 is configured to detect or to recognize an increase or modification of the sensing area 88.

Generally, it should be noted that the sensor segments 84, 85, 86 specifically mentioned herein exemplary represent the entirety of the sensor segments provided on the sensing surfaces 82, 83. The spatial resolution of the sensor assembly 18 may depend on the total number of sensor segments 84, 85, 86 and their arrangement on the sensing surfaces 82, 83.

Operation of the injection device 1 as illustrated in FIG. 8 may lead to two examples of a touch profile as indicated in FIGS. 11 and 12. Here, the user has a somewhat lateral contact to the trigger 9. At the beginning of a dose injection operation the thumb 114 of the user is in touch or contact with a lateral part of the sensing surface 82. Towards the center of the sensing surface 82 the contact strength or pressure decreases, as illustrated by the sensing area 89 and the sensor segment 85. As the dispensing procedure continues the dial extension 27 is subject to a distally directed movement, thus leading to a somewhat natural rolling motion of the thumb 114 across the surface of the sensing surface 82.

As illustrated in FIG. 12 and compared to the example of FIG. 11, the sensing area 88 representing a substantial mechanical contact moves towards the center of the sensing surface 82. Accordingly, the sensor segment 85, which detected a rather low pressure in the configuration of FIG. 11 now detects a comparatively large pressure in the configuration of FIG. 12.

The configuration as illustrated in FIG. 17 matches with the contact or touch profiles as illustrated in FIGS. 13 and 14. Here, from the start until the end of an injection the thumb 114 is located nearly in the center of the sensing surface 82. As the injection procedure continues or proceeds the different sensing areas 88, 89 representing a moderate and a high contact pressure only slightly increase or change their size, only moderately change their position and only slightly change their geometry as becomes apparent from a comparison of FIGS. 13 and 14.

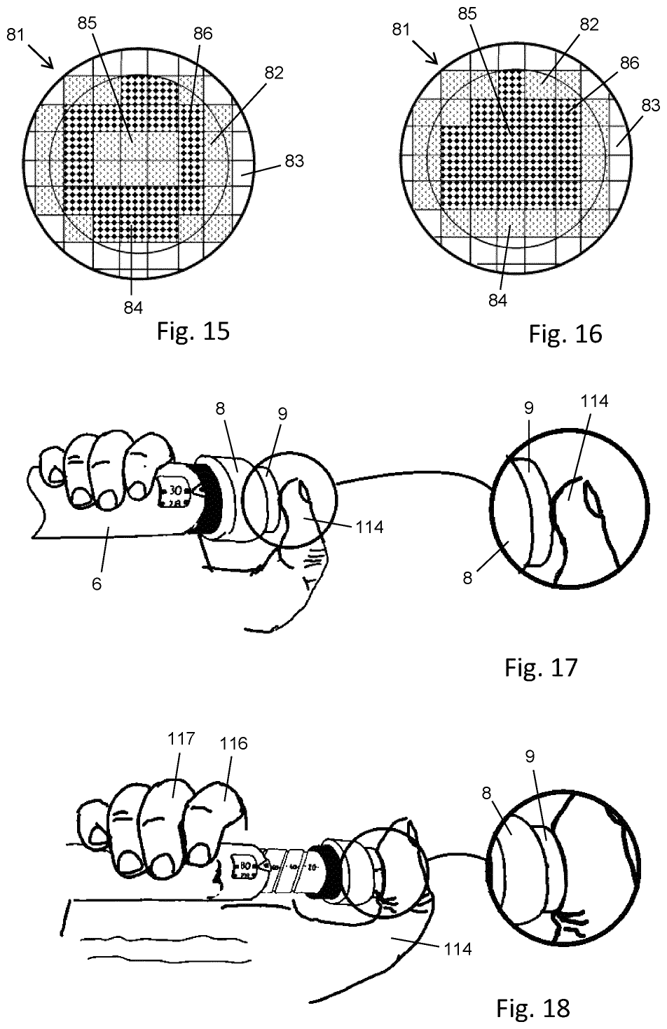

In another example, as e.g. illustrated in FIG. 18 and as represented by the pressure profiles of FIGS. 15 and 16, the user may entirely cover the sensing surface 82 with his thumb 114 even at the beginning of a dose dispensing action. As illustrated in FIG. 15 there may be provided different sensing areas 88, 89 of variable pressure across the sensing surface 82. As the user applies an increasing pressure, the pressure profile as illustrated in FIG. 15 may change towards a pressure profile as shown in FIG. 16. Here, the center area of the sensing surface 82 and provided with the sensor segment 85 may change from a moderate pressure level to a comparatively high pressure level.

The sensor assembly 80 is particularly configured to detect and to monitor the temporal variations of the contact pressure applied to the individual sensor segments 84, 85, 86. This way, the sensor assembly 80 and hence the processor 44 is operable to ascertain a sensing area 88, 89. The processor may be operable to detect and/or to distinguish between different sensing areas 88, 89, each included of one or numerous sensor segments 84, 85, 86.

The various sensing areas 88, 89 may distinguish from each other by the pressure levels applied to the respective sensing areas 88, 89. The processor 44 and/or the sensor assembly 80 may be further configured to detect or to characterize a movement of a sensing area 88, 89 in the course of e.g. a dose setting or dose dispensing procedure.

Moreover, the processor 44 and/or the sensor assembly 80 may be configured to detect or to characterize a variation of the size of the sensing areas 88, 89 as illustrated by the comparison of FIGS. 9 and 10 or 15 and 16.

Moreover, the sensor assembly 80 and/or the processor 44 may be operable to detect or to characterize a variation of a geometry or orientation of the sensing areas 88, 89 of the sensing surfaces 82, 83.

The temporal variation of a pressure profile or contact profile as indicated by FIGS. 9 and 10, by FIGS. 11 and 12, by FIGS. 13 and 14 and/or by FIGS. 15 and 16 may be indicative of one and the same or of different operation modes of the injection device 1.

The sensor assembly 80 and/or or processor 44 may be switchable into a training mode, thereby recording or capturing touch signal profiles over time and assigning such touch signal profiles with a specific operation of the injection device 1. In this way, the sensor assembly 80 and/or the processor 44 can be trained to a specific, e.g. a user-specific, touch signal profile that is associated or assigned with a specific operation of the injection device.

Later on and during use of the injection device 1, the sensor assembly 80 may be operable to detect temporal variations of electric touch signal that arises due to the user-induced operation of the injection device. The temporal variation of electric touch signals gathered or captured during operation of the injection device 1 by the sensor assembly 80 may be used to generate an operation profile. The operation profile may be then compared with numerous touch signal profiles previously stored in the memory 40 of the sensor assembly 80.

Then, the processor 44 may be configured to select one touch signal profile from the memory having a best match with the operation profile and to select the specific operation of the injection device assigned with the selected touch signal profile as a recognized operation of the injection device. In this way, and by analyzing and evaluating the temporal variation of the electric touch signal as provided by the sensor element 81, the sensor assembly 80 and/or the electronic module 34 may autonomously distinguish between different operation modes of an injection device.

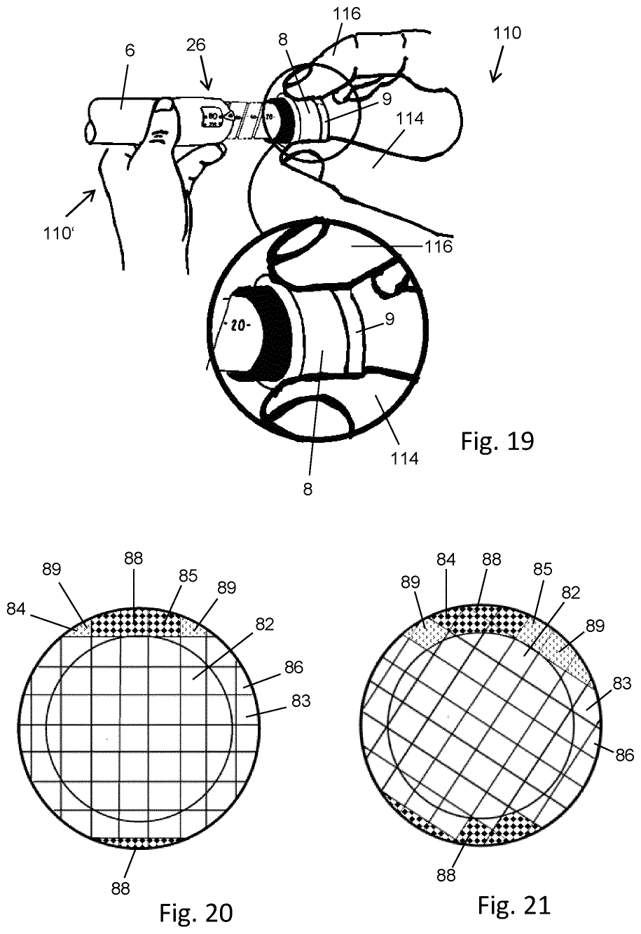

As particularly illustrated in FIGS. 20 and 21 in connection with FIG. 19, the user may use a thumb 114 and the index finger 116 to rotate a dose dial 8, which may be likewise provided with the tubular shaped sensing surface 83 of the sensor element 81. In FIG. 20 it is only the sensor segment 85 which is subject to a substantially high-pressure whereas the sensor segment 84 is subject to a lower or moderate pressure and the sensor segment 86 is not subject to any pressure at all. By rotating the dose dial 8, e.g. clockwise a pressure profile along or across the sensing surface 83 is subject to a gradual change. In the configuration of FIG. 20 the sensing areas 88, which coincide with the position of the thumb 114 and the index finger 116 are circumferentially framed or enclosed by sensing areas 89 of rather low pressure or force.

By rotating the dose dial 8 the sensor segment 84 is exposed to an increase of the contact pressure. As illustrated, the sensing areas 88, 89 move in circumferential direction with regard to the position of the sensor segment 84, 85, 86. This movement of the sensing areas 88, 89 can be detected and tracked as well as quantitatively measured by the processor 44. In this way, the sensing surface 83 and the individual sensor segments 84, 85, 86 also allow and support a quantitative measurement of a degree of rotation of the dose dial 8 relative to the body 6.

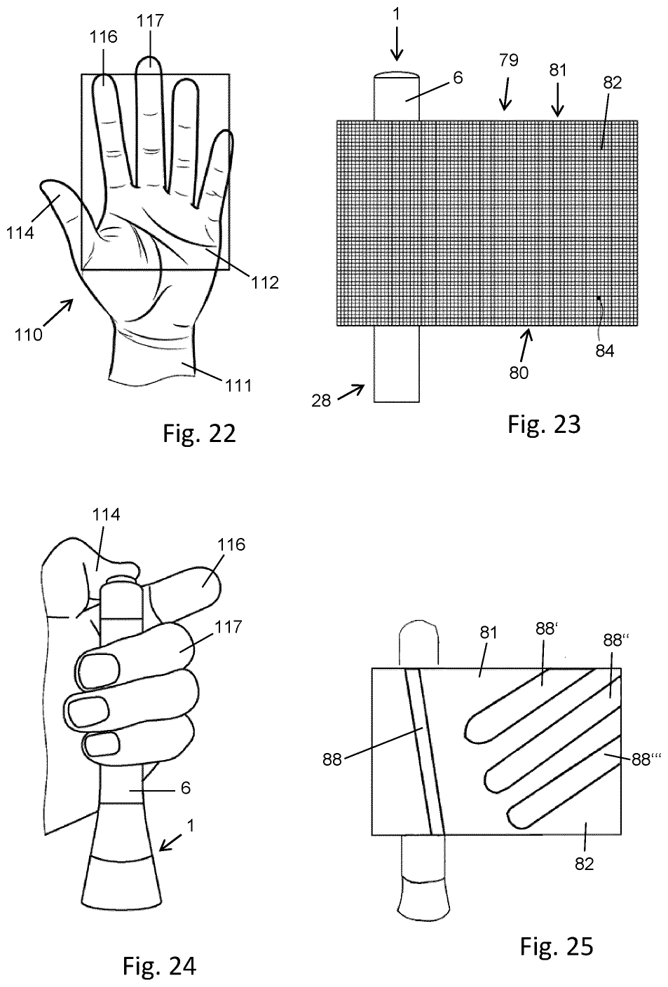

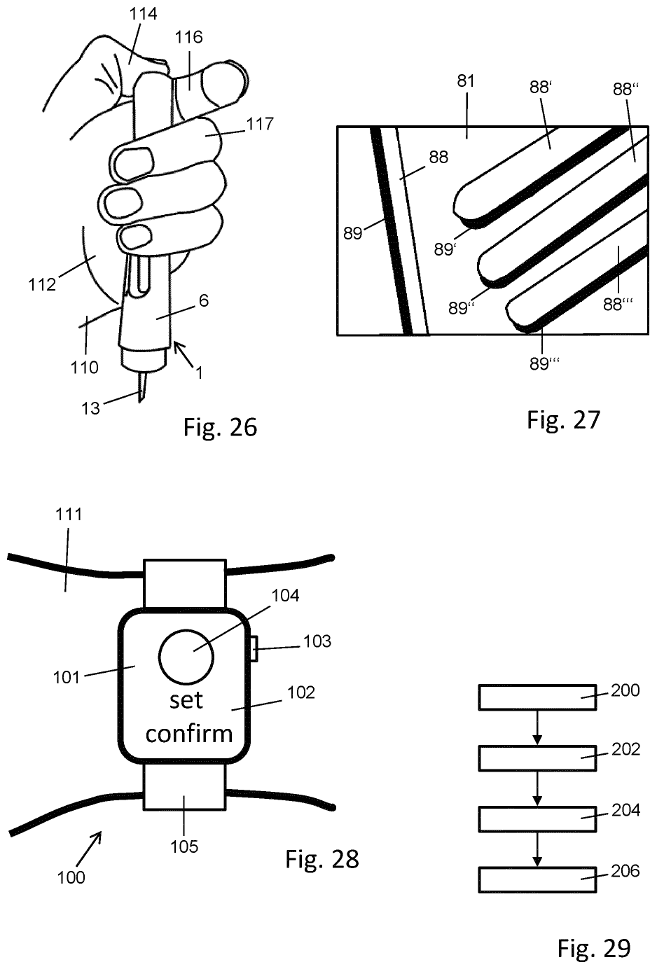

In the further illustration of FIGS. 22-27, the sensor assembly 80 includes a flexible sheet 79 configured to clasp around or to wrap around the body 6 of an injection device 1. There, the injection device 1 may be implemented as an autoinjector. The injection device 1 may be void of a separate trigger to be depressed e.g. by a thumb 114 of a user. Rather, a dose dispensing or dose injection operation may be simply triggered by bringing a distal dispensing end of the injection device 1 into contact with the skin and by applying a moderate pressure onto the skin via the housing 10 or body 6 of the injection device 1.

Then, the injection device 1 may automatically start a dispensing procedure by pushing the injection needle into the skin followed by a delivery or injection of the medicament into the skin portion which is actually punctured.

As it is illustrated in FIGS. 22-25 in a typical scenario of use and before applying a distally directed pressure onto the body 6 of the injection device a user may clasp around the body 6 of the injection device 1, which is provided with the sensor assembly 80 as described before. The sensor assembly 80 may include a flexible sheet 79 or foil with numerous sensor segments 84, 85, 86 allowing for a precise and spatially resolved recording and detection of a holding force or pressure applied by the fingers 114, 116, 117 and the palm 112 of the hand 110 of the user.

When a user holds the injection device 1 in his hand 110, the contact area between the palm 112 and the sensor element 81 may be represented by a sensing area 88 as illustrated in FIG. 25. The contact between the index finger 116 and the sensor element 81 may reflect in the sensing area 88′. The contact zone between the middle finger 117 and the sensor element 81 may be represented by the sensing area 88″ and the contact zone between a ring finger of the hand 110 and the sensor element 81 may be reflected by the sensing area 88′″.

Now and when the user brings the dispensing end of the injection device 1 in contact with the skin and starts to urge the injection device 1 against the skin a slight but measurable deformation in the contact area between the hand 110 and the sensor element 81 will arise. Since the hand 110 flexes along the longitudinal direction on the sensing surface 82 towards the distal end the sensor assembly 80 is operable to record a respective movement of the sensing areas 88, 88′, 88″, 88′″ towards sensing areas 89, 89′, 89″ and 89′″. The temporal variation and/or movement of the sensing areas 88, 89 can be recorded by the processor 44.

Moreover, and at the end of a dose dispensing procedure the processor 44 may be further operable to measure a duration during which the sensing areas 89, 89′, 89″, 89′″ remains substantially constant. In the same way, the sensor assembly 80 may be configured to detect how long the user keeps the thumb 114 firmly pressed on the trigger 9, e.g. at the end of a dose dispensing procedure. In this way, the sensor assembly 80 is configured to automatically detect and to record a prescribed holding time, during which the injection needle 13 should remain in the punctured tissue after termination of the dose injection of the medicament.

Upon termination of a dose injection procedure the sensor assembly 80 and/or electronic module 34 may be configured to provide a perceivable feedback to the user, either visually, acoustically or in a palpable way. For this, the onboard signal generators 51, 52 can be used or activated by the processor 44. Alternatively, and when the sensor assembly 80 is in a communication mode with the external electronic device 100 a respective feedback signal may be also generated by the external electronic device 100.

When the end of an injection procedure is reached, the user may be obliged to maintain a pressure on the trigger 9. This may lead to rather constant electric touch signals provided by the numerous touch sensitive sensor segments 84, 85, 86. Insofar, a rather constant and non-modifying pressure profile as measured by the sensor assembly 80 and simply followed by a complete release of the sensing surface 82, wherein the body part is disconnected from and is hence no longer in contact with the sensing surface 82, 83, is a direct indication that the user complied with the prescribed holding of the injection device after termination of the dose injection procedure.

Here, the sensor assembly is configured to automatically distinguish between different operation modes of the injection device. It may automatically record the time and/or the duration and which such different operations of the injection device took place.

In this way, the precision and quality of the data gathered by the sensor assembly can be improved.

FIG. 28 an example of an external electronic device 100 is schematically illustrated. The external electronic device 100 is implemented as a smartwatch. It includes a housing 101 and a display 102, which may be implemented as a touch sensitive display. The external device 100 may further include a control element 103 operable or actuatable by a user of the device. By way of a wristband 115 the external electronic device 100 can be attached to a wrist 111 of the user. On the display 102 there may be provided numerous visual items 104, in form of symbols, text or the like information by way of which the user may be assisted in using the injection device 1.

With a communication link between the sensor assembly 80 and the electronic module 34 it may be no longer necessary that the user confirms a certain operation mode of the injection device 1 manually with the external electronic device 100. Due to a communication link, e.g. a wireless communication link between the external electronic device 100 and the sensor assembly 80 the external electronic device 100 may be automatically provided with information such as setting of a dose at a certain point of time and dispensing of a dose at a certain point of time.

Moreover, the sensor assembly 80 may be further configured to detect or to quantitatively measure a size of a dose actually set by a user of the injection device. Upon detecting a dispensing procedure and by distinguishing a dispensing procedure from e.g. a dose setting procedure the sensor assembly 80 and the electronic module 34 may automatically provide the relevant information to the external electronic device 100, which may provide further data processing or data analysis. Moreover, the external electronic device 100 may be configured to transmit the acquired data to a healthcare provider for further data analysis and for controlling the user's compliance with a prescribed medication schedule.

The flowchart of FIG. 29 is illustrative of a method of recognizing, detecting, characterizing or measuring an operation of an injection device by making use of a sensor assembly 80 as described above. In a first step 200 a sensor element 81 of a sensor assembly 80 is either attached or integrated to or into an injection device 1 or to or into an add-on device 30 as described above. In a subsequent step 202 a number of electric touch signals are generated or modified in response to a body part of a user touching the sensing surface 82, 83 of the sensor element 81. In a subsequent step 204 a variation of electric touch signals is detected and/or or recorded over time. In a subsequent step 206 an operation of the injection device 1 is detected, recorded, characterized or quantitatively measured on the basis of the temporal variation of the electronic touch signals.

### REFERENCE NUMBERS

1 injection device
2 distal direction
3 proximal direction
4 dose incremention direction
5 dose decrementing direction
6 body
7 cartridge holder
8 dose dial
9 trigger
10 housing
11 connector
12 needle assembly
13 injection needle
14 inner needle cap
15 outer needle cap
16 protective cap
18 piston
19 piston rod
20 drive mechanism
21 medicament container
22 barrel
23 seal
24 medicament
25 outlet
26 window
27 dial extension
28 tubular member
30 add-on device
34 electronic module
36 printed circuit board
38 transceiver
39 transceiver
40 memory
42 clock
44 processor
46 power source
48 acceleration sensor
50 position sensor
51 signal generator
52 signal generator
53 light source
54 light source
55 window
56 window
60 device body
61 sidewall
62 flange portion
63 receptacle
64 receptacle
65 return element
66 recess
67 stop face
68 recess
69 stop face
70 support
71 stem
72 projection
74 projection
75 cover
76 end face
77 sidewall
79 sheet
80 sensor assembly
81 sensor element
82 sensing surface
83 sensing surface
84 sensor segment
85 sensor segment
86 sensor segment
88 sensing area
89 sensing area
100 external device
101 housing
102 display
103 control element
104 visual item
110 hand
111 wrist
112 palm
114 thumb
116 finger
117 finger

1-20. (canceled)

21. A sensor assembly for detecting an operation of an injection device, the sensor assembly comprising:
a sensor element attachable to the injection device and comprising a sensing surface, the sensing surface comprising a number of touch sensitive sensor segments, the touch sensitive sensor segments being operable to generate an electric touch signal when touched by a body part of a user,
a processor connectable to the sensor element, operable to detect a variation of the electric touch signals over time and to recognize an operation of the injection device at least partially on the basis of the temporal variation of the electric touch signals.

22. The sensor assembly according to claim 21, wherein at least one of the touch sensitive sensor segments is operable to generate different electric touch signals in response to a variation of a pressure applied to the touch sensitive sensor segment.

23. The sensor assembly according to claim 21, wherein each of the touch sensitive sensor segments is operable to generate or to modify an electric touch signal when touched by the body part of the of user.

24. The sensor assembly according to claim 21, wherein the processor is operable to process electric touch signals of a number of touch sensitive sensor segments to ascertain a sensing area of the sensing surface being touched by the body part.

25. The sensor assembly according to claim 24, wherein the processor is operable to detect a movement of the sensing area on the sensing surface.

26. The sensor assembly according to claim 24, wherein the processor is operable to detect a variation of a size of the sensing area on the sensing surface.

27. The sensor assembly according to claim 24, wherein the processor is operable to detect a variation of a geometry or orientation of the sensing area on the sensing surface.

28. The sensor assembly according to claim 21, wherein the sensor element comprises a planar sensing surface configured for fastening to an end face of a trigger of the injection device.

29. The sensor assembly according to claim 21, wherein the sensor element comprises a tubular shaped sensing surface configured for fastening to a tubular member of the injection device.

30. The sensor assembly according to claim 29, wherein the sensor element comprises a flexible sheet configured for wrapping around the tubular member of the injection device.

31. The sensor assembly according to claim 21, wherein the processor is operable to detect at least one of a dose setting operation of the injection device, a dose dispensing operation of the injection device and an end of dose holding operation of the injection device by processing numerous electric touch signals of numerous touch sensitive sensor segments over time when the sensor element is attached to the injection device.

32. The sensor assembly according to claim 31, wherein the processor is operable to distinguish between a dose setting operation, a dose dispensing operation and an end of the dose holding operation of the injection device by processing numerous electric touch signals of numerous touch sensitive sensor segment over time when the sensor element is attached to the injection device.

33. The sensor assembly according to claim 21, further comprising a clock connected to the processor, wherein the processor is operable to detect or to measure at least one of a point of time, at which a user operates the injection device and a duration during which a user operates the injection device.

34. The sensor assembly according to claim 21, further comprising a memory connected to the processor, wherein the processor is operable to store use related data in the memory, wherein the data contains at least one of: a point of time at which the injection device was operated, the duration during which the device was operated, a size of a dose of the medicament set of injected by the injection device.

35. The sensor assembly according to claim 34, wherein the processor is operable:
to detect a temporal variation of electric touch signals during an operation of the injection device and to generate an operation profile at least partially on the basis of the detected temporal variation of electric touch signals,
to compare the operation profile with numerous touch signal profiles stored in the memory,
to select one touch signal profile from the memory having a best match with the operation profile, and
to select the specific operation of the injection device assigned with the selected touch signal profile as a recognized operation of the injection device.

36. The sensor assembly according to claim 21, further comprising a clock and a memory connected to the processor, wherein the processor is operable:
to monitor a temporal variation of electric touch signals during a specific operation of the injection device, and
to store the temporal variation of the electric touch signals as a touch signal profile in the memory and
to assign the touch signal profile with the specific operation of the injection device.

37. An injection device for injecting a dose of a medicament, the injection device comprising:
a body to accommodate a drive mechanism operable to withdraw or to expel the medicament from a medicament container,
at least one of a dose dial and a trigger actuatable by a user for injecting the dose and
a sensor assembly attached to or integrated into at least one of the body, the dose dial and the trigger, the sensor assembly comprising:
a sensor element attachable to the injection device and comprising a sensing surface, the sensing surface comprising a number of touch sensitive sensor segments, the touch sensitive sensor segments being operable to generate an electric touch signal when touched by a body part of a user, and
a processor connectable to the sensor element, operable to detect a variation of the electric touch signals over time and to recognize an operation of the injection device at least partially on the basis of the temporal variation of the electric touch signals.

38. An add-on device configured for fastening to an injection device, the add-on device comprising a device body and a fastener for fastening the body to at least one of a body, a dose dial and a trigger of the injection device, wherein the add-on device comprises a sensor assembly, comprising:
a sensor element attachable to the injection device and comprising a sensing surface, the sensing surface comprising a number of touch sensitive sensor segments, the touch sensitive sensor segments being operable to generate an electric touch signal when touched by a body part of a user, and
a processor connectable to the sensor element, operable to detect a variation of the electric touch signals over time and to recognize an operation of the injection device at least partially on the basis of the temporal variation of the electric touch signals.
